(19) United States
(12) Patent Application Publication
(10) Pub. No.: US 2016/0199354 A1
(43) Pub. Date: Jul. 14, 2016

(54) METHODS FOR OLMESARTAN DOSING BY AUC

(71) Applicant: Autotelic LLC, City of Industry, CA (US)

(72) Inventor: Vuong Trieu, Agoura Hills, CA (US)

(73) Assignee: Autotelic LLC, City of Industry, CA (US)

(21) Appl. No.: 14/992,898

(22) Filed: Jan. 11, 2016

Related U.S. Application Data
(60) Provisional application No. 62/101,895, filed on Jan. 9, 2015.

(51) Int. Cl. A61K 31/4178; A61K 45/06; G01N 33/94; A61K 31/505

(52) U.S. Cl. CPC A61K 31/4178; A61K 31/505; A61K 45/06; G01N 33/9453

(57) ABSTRACT

Methods for antihypertensive drug dosing by pharmacokinetic parameter determination. In one embodiment, the antihypertensive drug is olmesartan and the pharmacokinetic parameter is AUC. The methods are effective for treating hypertension.

### CROSS-REFERENCE TO RELATED APPLICATION

This applications claim the benefit of U.S. Application No. 62/101,895, filed Jan. 9, 2015, expressly incorporated herein by reference in its entirety.

### BACKGROUND OF THE INVENTION

Today hypertension affects approximately one billion people worldwide and is projected to increase to 1.38 billion people by 2019. Hypertension is a condition that is regarded as a risk factor that progresses heart diseases and ultimately causes adverse cardiac symptoms.

Blood pressure control can often be achieved by antihypertensive therapy with one or more drugs. However, the effectiveness of treating hypertension with antihypertensive drug therapy can vary greatly due to variations from individual to individual. Consequently, there is high variability of the pharmacokinetics for antihypertensive drugs when conventionally dosed at mg/day, which in turn results in highly variable blood pressure reduction. As a result, conventional dosing leads to a poor correlation between dose and blood pressure reduction and overall effectiveness of treatment.

Despite advances in the development of antihypertensive drugs, their formulations, and methods for their administration, a need exists to further improve the clinical effectiveness of the administration of antihypertensive drugs. The present invention seeks to fulfill this need and provides further related advantages.

### SUMMARY OF THE INVENTION

In one aspect, the invention provides a method for olmesartan dosing by AUC. In one embodiment, the method comprises:

(a) administering olmesartan at a first dose to a subject in need of hypertension therapy;

(b) determining the concentration of olmesartan the subject's blood at one or more time points after administration to provide a set of olmesartan concentration/time data points;

(c) transforming the set of olmesartan concentration/time data points to provide area-under-the-curve (AUC); and

(d) administering olmesartan at subsequent doses (e.g., second and subsequent doses) to achieve a target AUC of about 7,000 hr*ng/mL.

In certain embodiments, the target AUC is 7,000 hr*ng/mL +/−5%. In other embodiments, the target AUC is 7,000 hr*ng/mL +/−2%. In further embodiments, the target AUC is 7,000 hr*ng/mL +/−1%. In still other embodiments, the target AUC is 7,000 hr*ng/mL +/−0.5%.

In another aspect, the invention provides a method for antihypertensive drug dosing by one or more pharmacokinetic parameters. In one embodiment, the method comprises:

(a) administering an antihypertensive drug at a first dose to a subject in need of hypertension therapy;

(b) determining the concentration of the antihypertensive drug the subject's blood at one or more time points after administration to provide a set of antihypertensive drug concentration/time data points;

(c) transforming the set of antihypertensive drug concentration/time data points to provide one or more pharmacokinetic parameters; and

(d) administering the antihypertensive drug at subsequent doses (e.g., second and subsequent doses) to achieve a target optimal value for the one or more pharmacokinetic parameters.

The one or more pharmacokinetic parameters can be one or more of concentration time course, peak concentration (Cₘₐₓ), and time after administration to peak concentration, terminal half-life, area-under-the-curve (AUC), bioavailability, absorption, distribution, metabolism, excretion, biotransformation, and combinations thereof. In one embodiment, the pharmacokinetic parameter is area-under-the-curve (AUC).

In certain embodiments, the target optimal value is +/−5% of the target optimal value. In other embodiments, the target optimal value is +/−2% of the target optimal value. In further embodiments, the target optimal value is +/−1% of the target optimal value. In still other embodiments, the target optimal value is +/−0.5% of the target optimal value.

In certain embodiments of the above methods, the second dose is the same or substantially the same as the first dose; in other embodiments, the second dose is greater than the first dose; and in further embodiments, the second dose is less than the first dose.

In certain embodiments, the above methods further including repeating steps (a)-(d) until blood pressure control is achieved.

In certain embodiments of the above methods, the subject is in need of treatment for hypertension and dyslipidemia, and the method comprises administration of olmesartan (or an antihypertensive drug) and an anti-dyslipidemia drug.

In certain embodiments of the above methods, the subject is in need of treatment for resistant hypertension. In certain of these embodiments, the subject was previously treated for hypertension with a three-drug regimen comprising a first drug, a second drug, and a third drug, wherein the first drug is a diuretic, and wherein the subject's blood pressure remained elevated above an established blood pressure goal following the three-drug regimen.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 demonstrates individual values of olmesartan AUC when dosed as either olmesartan single agent (OM) or olmesartan/rosuvastatin (OM/RC).

FIG. 2 demonstrates the variability of systolic blood pressure (SBP) reduction after dosing with olmesartan single agent (olmesartan) or olmesartan/rosuvastatin fixed dose combination (FDC) (ST-101).

FIG. 3 demonstrates the variability of diastolic blood pressure (DBP) reduction after dosing with olmesartan single agent (olmesartan) or olmesartan/rosuvastatin fixed dose combination (FDC) (ST-101).

FIG. 4 demonstrates the dose response to systolic blood pressure (SBP) reduction after dosing with olmesartan single agent (olmesartan) (non-Asian subjects solid circles) (Asian subjects open circles), or olmesartan/rosuvastatin fixed dose combination (OM/RC FDC) (open triangle).

FIG. 5 demonstrates the dose response to diastolic blood pressure (DBP) reduction after dosing with olmesartan single agent (olmesartan) (non-Asian subjects solid circles) (Asian subjects open circles), or olmesartan/rosuvastatin fixed dose combination (OM/RC FDC) (open triangle).

FIG. 6 demonstrates the linear correlation between the first and second pharmacokinetic determinations (AUC) for olmesartan.

FIG. 7 demonstrates the linear correlation between dose and olmesartan (OM) AUC after dosing with olmesartan single agent (olmesartan) (non-Asian subjects solid circles) (Asian subjects open circles), or olmesartan/rosuvastatin fixed dose combination (OM/RC FDC) (open triangle).

FIG. 8 demonstrates the dose proportionality between dose and olmesartan AUC after dosing with olmesartan single agent (olmesartan) (non-Asian subjects solid circles) (Asian subjects open circles), or olmesartan/rosuvastatin fixed dose combination (OM/RC FDC) (open triangle).

### DETAILED DESCRIPTION OF THE INVENTION

In one aspect, the invention provides methods for treating hypertension by hypertensive, in general, and olmesartan or olmesartan/rosuvastatin FDC, in specific. In the method, a dosing regimen targeting specific AUC is provided in which AUC determined from first dosing with an antihypertensive drug is used to adjust subsequent dosing to achieve the targeted AUC. The targeted AUC dosing regimen for olmesartan was made possible by our discovery of: (1) the targeted AUC value derived from our olmesartan pharmacokinetic studies; (2) the ability to predict subsequent AUCs by AUC after first dose; and (3) the method of adjustment taking advantage of our demonstration of olmesartan dose proportionality when dosed as either olmesartan or as olmesartan/rosuvastatin FDC.

In certain embodiments, the pharmacokinetic parameter used in the method is area-under-the-curve (AUC).

The methods of the invention are effective in treating resistant hypertension.

As noted above, antihypertensive drug pharmacokinetic variability results in variability in hypertensive therapy effectiveness due to poor dose response. In the methods of the present invention, determination of drug exposure as AUC allows for adjustment of subsequent dosing because of the observed linear correlation was found between antihypertensive drug dose and the AUC.

Pharmacokinetic Variability

Antihypertensive drug pharmacokinetic variability was determined for olmesartan, a representative antihypertensive drug.

Drug exposure as AUC was studied among Korean healthy males aged between 20 and 50 years in two clinical studies: (1) a randomized, open-label, multiple-dose, cross-over study of 36 subjects divided into 6 cohorts and treated over 3 periods (subjects were administered either 1 tablet of 40 mg olmesartan medoxomil, 1 tablet of 20 mg rosuvastatin calcium, or a 1 tablet each of both agents (fixed dosage composition, single dose form) orally every 24 hours for 7 days, with an 8-day washout period separating the individual treatment courses) and (2) a randomized, open-label, single-dose, cross-over study of 58 subjects divided into 2 cohorts and treated over 2 periods (Son H, Roh H, Lee D, Chang H, Kim J, Yun C, et al. Pharmacokinetics of rosuvastatin/olmesartan fixed-dose combination: a single-dose, randomized, open-label, 2-period crossover study in healthy Korean subjects. Clin Ther 2013; 35:915-22) (one tablet of ST-101, also known as olmesartan/rosuvastatin FDC, tablet of 40 mg olmesartan medoxomil/20 mg rosuvastatin calcium was administered once orally as the test drug, or 1 tablet each of 40 mg olmesartan medoxomil and 20 mg rosuvastatin calcium was co-administered once orally as the comparator; eligible subjects were randomly assigned to 2 groups (test arm vs comparator arm) in a 1:1 ratio; each subject received a single dose of the FDC or the comparator orally in a fasted state, with a 7-day washout period between the administrations.

A total of 34 volunteers in study 1 and 54 volunteers in study 2 were evaluated for olmesartan pharmacokinetic (PK) analysis. For PK analyses, blood samples were collected at 0 (pre-dose), 0.5, 2, 2.5, 3, 4, 5, 6, 7, 8, 10, 12, 16, 24, 48 hour after dosing of olmesartan either as FDC or as single agent.

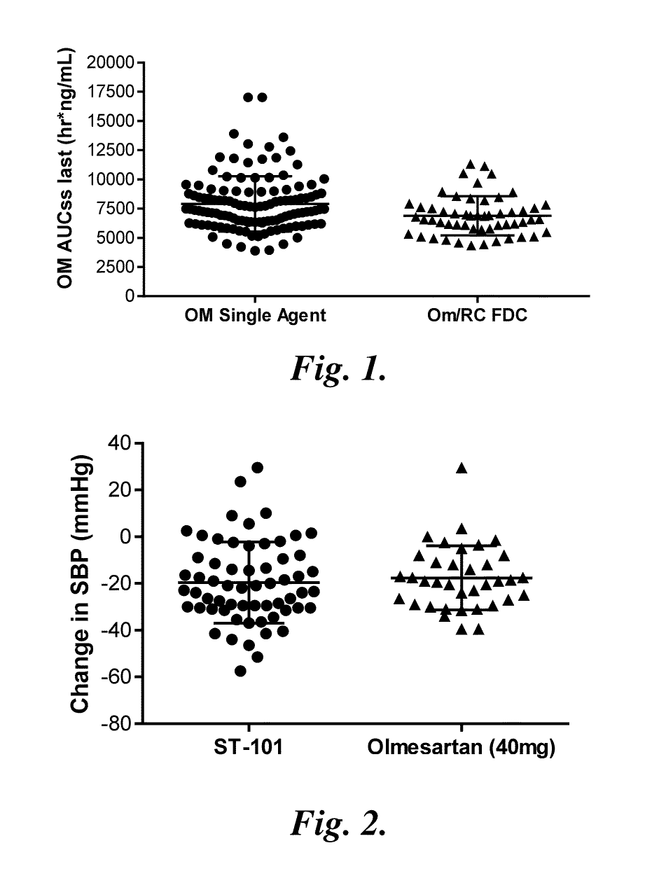

FIG. 1 compares individual values of olmesartan AUC when dosed as either olmesartan single agent (OM) or olmesartan/rosuvastatin (OM/RC) fixed dose composition (FDC). As shown in FIG. 1, olmesartan exhibited wide AUC variabilities with non-Gaussian distribution. The coefficient of variations were 29.95% and 24.42% for olmesartan single agent and OM/RC FDC, respectively. The tabulated summary of the data including Gaussian tests are shown below in Table 1.

TABLE 1

| | OM Single Agent | OM/RC FDC |
|---|---|---|
| Number of values | 122 | 54 |
| Minimum | 3899 | 4342 |
| 25% Percentile | 6317 | 5752 |
| Median | 7450 | 6695 |
| 75% Percentile | 8916 | 7680 |
| Maximum | 17026 | 11329 |
| Mean | 7908 | 6892 |
| Std. Deviation | 2369 | 1683 |
| Std. Error of Mean | 214.5 | 229.1 |
| Lower 95% CI of mean | 7483 | 6432 |
| Upper 95% CI of mean | 8332 | 7351 |

D′Agostino & Pearson omnibus normality test

| | | |
|---|---|---|
| K2 | 37.27 | 6.992 |
| P value | <0.0001 | 0.0303 |

Passed normality test

| | | |
|---|---|---|
| (alpha = 0.05) | No | No |
| P value summary | **** | * |

Shapiro-Wilk normality test

| | | |
|---|---|---|
| W | 0.9087 | 0.9407 |
| P value | <0.0001 | 0.0099 |

Passed normality test

| | | |
|---|---|---|
| (alpha = 0.05) | No | No |
| P value summary | **** | ** |

Hypertension Therapy Effectiveness Variability

Hypertension therapy effectiveness was evaluated by evaluating the variability of systolic blood pressure (SBP) and diastolic blood pressure (DBP) reduction following dosing with olmesartan single agent (OM) or a fixed dosage composition comprising olmesartan and rosuvastatin (OM/RC FDC).

The high variability of olmesartan PK resulted in the high variability of blood pressure response with olmesartan single agent OM or OM/RC FDC. The study was a multi-site, randomized, double-blind, placebo-controlled, factorial, 4 arm, 8 week Phase 3 clinical study of Korean subjects with hypertension associated with dyslipidemia aged between 20 and 80 years. Following a 4-week therapeutic lifestyle-change period that included a washout period, eligible patients were randomized in a 2:1:1:1 ratio to receive either OM/RC FDC, olmesartan medoxomil 40 mg, rosuvastatin calcium 20 mg, or placebo, once daily, for up to 8 weeks.

A total of 183 subjects were randomized (72 subjects in OM/RC FDC, 39 subjects in olmesartan medoxomil 40 mg, 38 subjects in rosuvastatin calcium 20 mg, and 34 subjects in placebo). Among the 183 randomized subjects, 181 subjects took the investigational drug (ST-101/OM/RC FDC), 162 subjects completed the study, and 21 of 162 subjects were discontinued from the study.

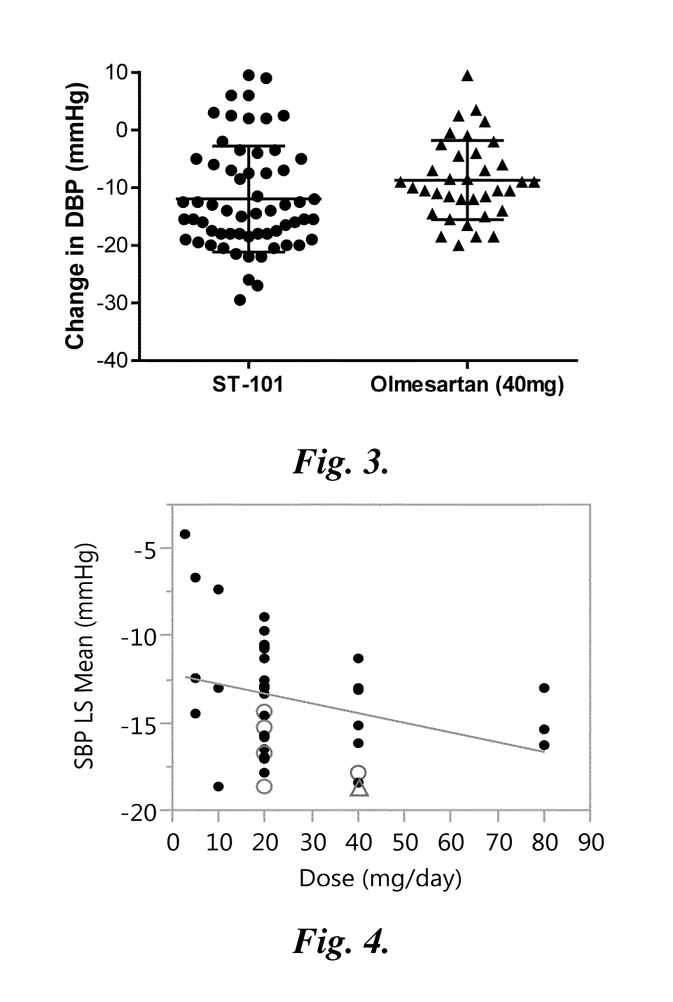

The SBP and DBP reduction was highly variable. FIG. 2 compares the variability of systolic blood pressure (SBP) reduction after dosing with olmesartan single agent (olmesartan) or olmesartan/rosuvastatin fixed dose combination (OM/RC FDC, ST-101). FIG. 3 compares the variability of diastolic blood pressure (DBP) reduction after dosing with olmesartan single agent (olmesartan) or olmesartan/rosuvastatin fixed dose combination (OM/RC FDC, ST-101). The coefficient of variation for SBP reduction was 88.39% and 78.24% for OM/RC FDC and OM, respectively; and the coefficient of variation for DBP reduction was 76.78% and 79.25% for OM/RC FDC and OM, respectively. It is likely that about half of the variability is coming from the PK variability and the remainder is likely from individual variability as to response. The distribution of SBP and DBP reduction is graphically presented below.

Antihypertensive Drug Dose Response Correlation

Antihypertensive drug dose to blood pressure reduction response was determined to be poor.

The high variability of PK and the resulting pharmacodynamics (PD) of olmesartan resulted in weak correlation between dose and blood pressure reduction. PubMed database was searched to identify relevant scientific and review articles from June 2001 to June 2015 using the search terms olmesartan efficacy, response, effect/clinical trial. Reference lists of included articles were reviewed to locate additional references of interest. PD studies with olmesartan medoxomil dose range from 5 to 80 mg monotherapy were selected. PD response and demographic data from a total of 10,770 patients with hypertension in 21 clinical studies were included in the analysis (Oparil S, Williams D, Chrysant S G, Marbury T C, Neutel J. (2001). Comparative efficacy of olmesartan, losartan, valsartan, and irbesartan in the control of essential hypertension. J Clin Hypertens (Greenwich) 2001; 3(5):283-318; Püchler K, Laeis P, Stumpe K O. Blood pressure response, but not adverse event incidence, correlates with dose of angiotensin II antagonist. J Hypertens Suppl 2001 June; 19(1):S41-8; Neutel J M, Elliott W J, Izzo J L, Chen C L, Masonson H N. Antihypertensive efficacy of olmesartan medoxomil, a new angiotensin II receptor antagonist, as assessed by ambulatory blood pressure measurements. J Clin Hypertens (Greenwich) 2002(5):325-31; Stumpe, KO and Ludwig M. Antihypertensive efficacy of olmesartan compared with other antihypertensive drugs. J Hum Hypertens 2002; 16(Suppl 2):S24-8; Brunner H R, Stumpe K O, Januszewicz A. Antihypertensive efficacy of olmesartan medoxomil and candesartan cilexetil assessed by 24-hour ambulatory blood pressure monitoring in patients with essential hypertension. Clin Drug Investig 2003; 23(7):419-30; Liau C S, Lee C M, Sheu S H, Ueng K C, Chien K L, Su T C, et al. Efficacy and safety of olmesartan in the treatment of mild-to-moderate essential hypertension in Chinese patients. Clin Drug Investig 2005; 25(7):473-9; Destro M, Scabrosetti R, Vanasia A, Mugellini A. Comparative efficacy of valsartan and olmesartan in mild-to-moderate hypertension: results of 24-hour ambulatory blood pressure monitoring. Adv Ther 2005; 22(1):32-43; Smith D H, Dubiel R, Jones M. Use of 24-hour ambulatory blood pressure monitoring to assess antihypertensive efficacy. Am J Cardiovasc Drugs 2005; 5(1):41-50; Bohm M and Ewald S. Blood pressure reduction with olmesartan in mild-to-moderate essential hypertension: a planned interim analysis of an open label sub-study in German patients. Curr Med Res Opin 2006; 22(7):1375-80; Brunner H R and Arakawa K. Antihypertensive efficacy of olmesartan medoxomil and candesartan cilexetil in achieving 24-hour blood pressure reductions and ambulatory blood pressure goals. Clin Drug Investig 2006; 26(4):185-93; Chrysant S G, Marbury T C, Silfani T N. Use of 24-h ambulatory blood pressure monitoring to assess blood pressure control: a comparison of olmesartan medoxomil and amlodipine besylate, Blood Press Monit 2006; 11(3):135-41; Barrios V, Boccanelli A, Ewald S, Girerd X, Heagerty A, Krzesinski J M, et al., Efficacy and tolerability of olmesartan medoxomil in patients with mild to moderate essential hypertension: the OLMEBEST Study. Clin Drug Investig 2007; 27(8):545-58; Giles T D, Oparil S, Silfani T N, Wang A, Walker J F. Comparison of increasing doses of olmesartan medoxomil, losartan potassium, and valsartan in patients with essential hypertension. J Clin Hypertens (Greenwich) 2007; 9(3):187-95; Izzo J L, Neutel J M, Silfani T, Dubiel R, Walker F. Efficacy and safety of treating stage 2 systolic hypertension with olmesartan and olmesartan/HCTZ: results of an open-label titration study. J Clin Hypertens (Greenwich) 2007; 9(1):36-44; Oparil S, Chrysant S G, Kereiakes D, Xu J, Chavanu K J. Waverczak W, et al. Results of an olmesartan medoxomil—based treatment regimen in hypertensive patients. J Clin Hypertens (Greenwich) 2008; 10(12):911-21; Kereiakes D J, Neutel J, Stoakes K A, Waverczak W F, Xu J, Shojaee A, et al. The effects of an olmesartan medoxomil—based treatment algorithm on 24-hour blood pressure levels in elderly patients aged 65 and older. J Clin Hypertens (Greenwich) 2009; 11(8):411-21; Rana R and Singh A. Olmesartan medoxomil evaluated for safety and efficacy in Indian patients with essential hypertension: a real world observational post marketing surveillance. J Assoc Physicians India 2010; 58:77-83; Chrysant S G. Safety and Tolerability of an Olmesartan Medoxomil-Based Regimen in Patients with Stage 1 Hypertension. Clin Drug Investig 2010; 30(7):473-82; Oparil S and Pimenta E. Efficacy of an olmesartan medoxomil—based treatment algorithm in patients stratified by age, race, or sex. J Clin Hypertens (Greenwich) 2010; 12(1):3-13; Wang J G, Sun N L, Ke Y N, Zhang B H, Ikegami N, Zhu J R. Long-term efficacy of olmesartan medoxomil in Chinese hypertensive patients as assessed by clinic, ambulatory and home blood pressure measurements. Clin Drug Investig 2012; 32:729-734.

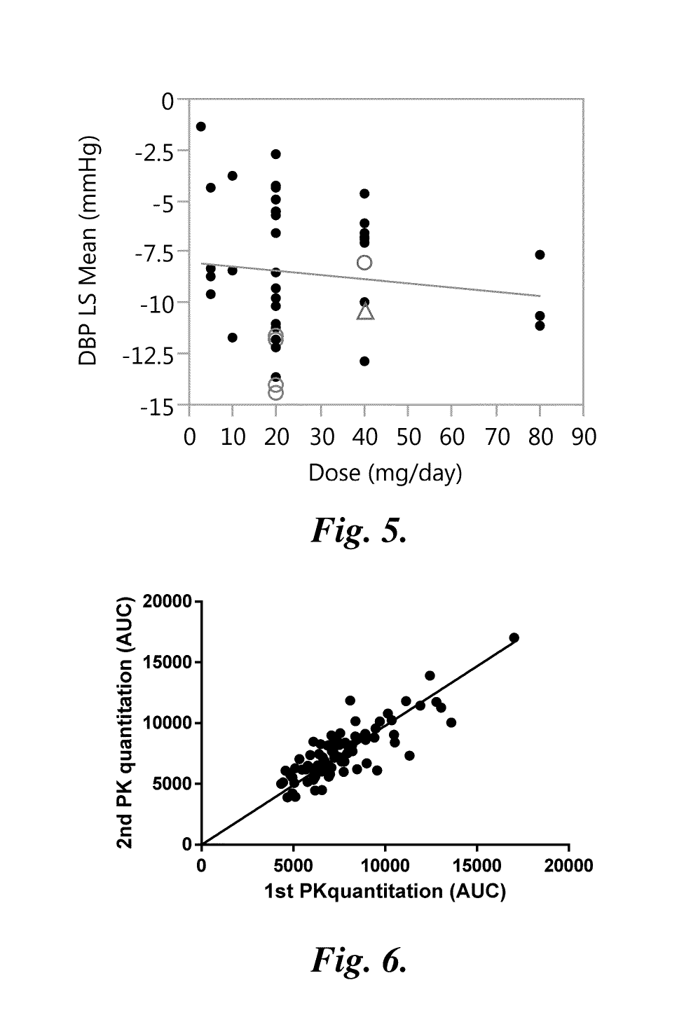

The mean reduction in DBP and SBP in patients from each study was plotted versus dose. FIG. 4 compares the dose response to systolic blood pressure (SBP) reduction after dosing with olmesartan single agent (olmesartan) (non-Asian subjects solid circles) (Asian subjects open circles), or olmesartan/rosuvastatin fixed dose combination (OM/RC FDC) (open triangle). FIG. 5 compares the dose response to diastolic blood pressure (DBP) reduction after dosing with olmesartan single agent (olmesartan) (non-Asian subjects solid circles) (Asian subjects open circles), or olmesartan/rosuvastatin fixed dose combination (OM/RC FDC) (open triangle). DBP and SBP reduction effects by ST-101 (OM/RC FDC) are similar to the effects of 40 mg of olmesartan in other studies. Regression analysis showed a very shallow insignificant relationship between DBP reduction and dose and only weak correlation between SBP reduction and dose (p=0.04).

Determination of First Pharmacokinetic Parameter and Prediction of Subsequent Pharmacokinetic Parameter

There is no known natural law to predict antihypertensive drug (e.g., olmesartan) pharmacokinetic parameters (e.g., AUC). The complexity of pharmacokinetics precludes any predictive methods for the accurate prediction of human olmesartan pharmacokinetics. The only means of predicting the pharmacokinetic parameters is to actually perform the pharmacokinetic study, which is shown herein to be predictive of subsequent pharmacokinetic parameter determination.

FIG. 6 illustrates the linear correlation between the first and second pharmacokinetic determinations (AUC) for olmesartan. The linear regression is described by the following equation: Y=0.9782*X. The p value of significant deviation from 0 is less than 0.0001.

Antihypertensive Drug Dose Proportionality

Antihypertensive drug dose proportionality was demonstrated.

Despite pharmacokinetic variability, when the mean AUC was taken into consideration, dose proportionality of olmesartan pharmacokinetics was demonstrated. PubMed database was searched to identify relevant scientific and review articles from May 2001 to June 2015 using the search term olmesartan PK. Reference lists of included articles were reviewed to locate additional references of interest. PK studies with single dose AUC of olmesartan medoxomil in healthy subjects were selected. Olmesartan medoxomil AUC from co-administration of olmesartan medoxomil with other drugs or FDC containing olmesartan medoxomil were also included if there was no known drug-drug interaction (DDI). The pharmacokinetic datasets for olmesartan were obtained from published clinical studies across Asian and non-Asian subjects with a dose range from 10 to 160 mg. More than 80% of the studies reported AUC in arithmetic mean. Thus, to make comparable analysis, only studies with AUC calculated by arithmetic mean were included in this analysis. PK and demographic data from a total of 895 healthy subjects from 13 clinical studies were included in the final analysis Schwocho L R and Masonson H N. Pharmacokinetics of CS866, a new angiotensin II receptor block, in healthy subjects. J Clin Pharmacol 2001; 41:515-27; von Bergmann K, Laeis P, Pilchler K, Sudhop T, Schwocho L R, Gonzalez L. Olmesartan medoxomil: influence of age, renal and hepatic function on the PK of olmesartan medoxomil. J Hypertens Suppl 2001 June; 19(1):S33-40; Takanori T and Ryuji U. Pharmacokinetics of repeated oral dosages of CS-866 (olmesartan medoxomil), angiotensin II receptor antagonist, planned to be used clinically. J Clin Ther Med (Japanese) 2003; 19:1143-56; Suwannakul S, Ieiri I, Kimura M, Kawabata K, Kusuhara H, Hirota T, et al. Pharmacokinetic interaction between pravastatin and olmesartan in relation to SLCO1B1 polymorphism. J Hum Genet 2008; 53(10):899-904; Rohatagi S, Lee J, Shenouda M, Haworth S, Bathala M S, Allison M, et al. Pharmacokinetics of amlodipine and olmesartan after administration of amlodipine besylate and olmesartan medoxomil in separate dosage forms and as a fixed-dose combination. J Clin Pharmacol 2008 November; 48(11):1309-22; Jiang J, Liu D, Hu P. Pharmacokinetic and safety profile of olmesartan medoxomil in healthy Chinese subjects after single and multiple administrations. Pharmazie 2009 May; 64(5):323-6; Li K Y, Liang J P, Hu B Q, Qiu Y, Luo C H, Jiang Y, et al. The relative bioavailability and fasting pharmacokinetics of three formulations of olmesartan medoxomil 20-mg capsules and tablets in healthy Chinese male volunteers: an open-label, randomized-sequence, single-dose, three-way crossover study. Clin Ther 2010; 32(9):1674-80; Chen X, Hu P, Jiang J, Liu T, Zhong W, Liu H, et al. Pharmacokinetic and pharmacodynamic profiles of a fixed-dose combination of olmesartan medoxomil and amlodipine in healthy Chinese males and females. Clin Drug Investig 2012; 32(12):783-90; Liu D Y, Jiang J, Wang C G, Zhang J Y, Hu P. Pharmacokinetics and tolerability of olmesartan medoxomil plus hydrochlorothiazide combination in healthy Chinese subjects: drug-drug interaction, bioequivalence, and accumulation. Int J Clin Pharmacol Ther 2014; 52(4):321-7; Jin C, Jeon J Y, Im Y J, Jung J A, Kim Y, Park K, et al. Pharmacokinetic properties and bioequivalence of olmesartan medoxomil/hydrochlorothiazide in healthy Korean male subjects. Int J Clin Pharmacol Ther 2014 January; 52(1):64-72; He L, Wickremasingha P, Lee J, Tao B, Mendell-Harary J, Walker J, et al. The effects of colesevelam HCl on the single-dose pharmacokinetics of glimepiride, extended-release glipizide, and olmesartan medoxomil. J Clin Pharmacol 2014 January; 54(1):61-9; Lee S Y, Kim J R, Jung J A, Huh W, Bahng M Y, Ko J W. Bioequivalence evaluation of two amlodipine salts, besylate and orotate, each in a fixed-dose combination with olmesartan in healthy subjects. Drug Des Devel Ther 2015 Jun. 2; 9:2811-7, reported PK information for Korean population.

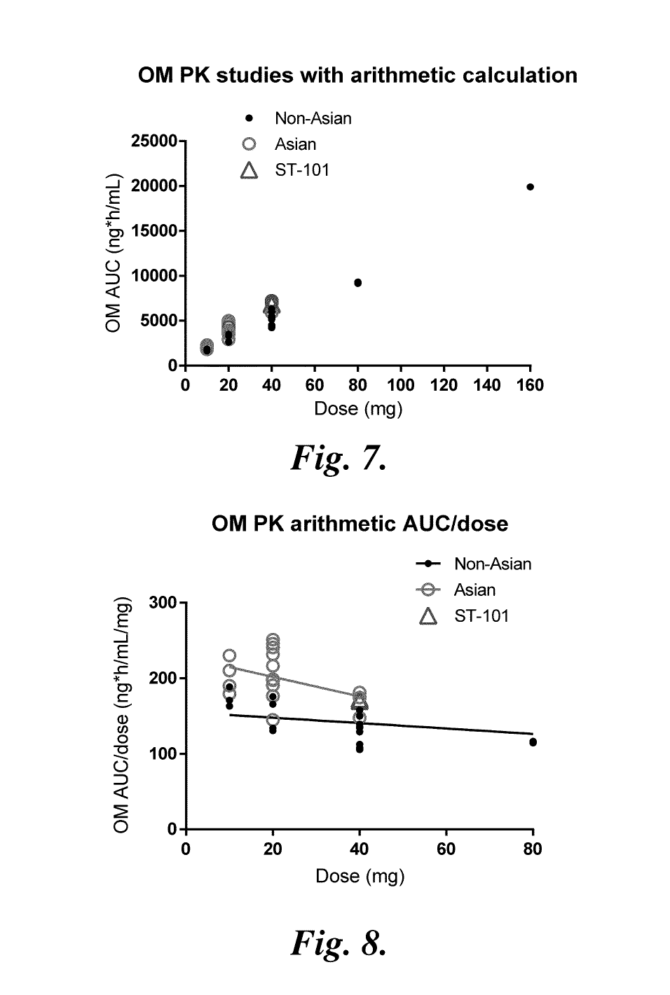

Dose proportional analysis was performed to confirm dose proportional nature of olmesartan PK. The mean AUCₗₐₛₜ of olmesartan from each study was plotted versus dose. FIG. 7 compares the linear correlation between dose and olmesartan (OM) AUC after dosing with olmesartan single agent (olmesartan) (non-Asian subjects solid circles) (Asian subjects open circles), or olmesartan/rosuvastatin fixed dose combination (OM/RC FDC) (open triangle). FIG. 8 compares the dose proportionality between dose and olmesartan AUC after dosing with olmesartan single agent (olmesartan) (non-Asian subjects solid circles) (Asian subjects open circles), or olmesartan/rosuvastatin fixed dose combination (OM/RC FDC) (open triangle).

Olmesartan AUC among Asian subjects is comparable to AUC in non-Asian subjects at the same dose level. Similarly, olmesartan AUC from ST-101 (OM/RC FDC) overlapped with both Asian and non-Asian subjects at 40 mg. The data shows linear dose-dependent increase in AUC from 10 to 160 mg of olmesartan. Dose proportional analysis was performed by demonstrating constant AUC/dose across doses. The dose-normalized AUC was plotted versus dose. The linear regression line of AUC/dose against dose was horizontal with 95% confidence intervals (CIs) of slopes close to 0 for both Asian (−2.93 to 0.34; p=0.11) and non-Asian groups (−0.65 to −0.06; p=0.02). The higher observed AUC for Asian was due the smaller body weight of this ethnic group.

Antihypertensive Drug Dosing by AUC

Given that subsequent pharmacokinetic parameters (e.g., AUC) can be predicted by first pharmacokinetic parameter quantitation and because there is dose proportionality, the method of the invention provides a method that improves the delivery of antihypertensive drugs in general and olmesartan or olmesartan/rosuvastatin FDC in particular.

In a representative method, olmesartan is administered to a subject to achieve AUC of 7,000 hr*ng/mL, which is the median of conventional olmesartan PK (AUC) at 40 mg/day dosing (see pharmacokinetic variability above). If dosing is below or above that AUC, dose adjustment can be adjusted up or down, respectively, as there is dose proportionality (e.g., if AUC is over 25%, the dose is reduced by 25%; if AUC is under by 40%, the dose is increased by 40%).

The target AUC dosing can be varied to higher or lower target AUC when the patient is demonstrating resistance or sensitivity, respectively, to the antihypertensive drug. Regardless, once the targeted AUC for the patient has been defined, the target AUC needed to be maintained despite changes in physical condition (weight) and physiological condition (kidney, liver functional status etc.). Variables such as weight and organ function factors are known to affect olmesartan pharmacokinetics. However, with frequent monitoring, it is possible to keep dosing at constant AUC by appropriate adjustment of dosing when changes are noted.

The methods of the invention effectively administer antihypertensive drugs (e.g., olmesartan) by pharmacokinetic parameter (e.g., AUC) dosing to avoid the problems associated with pharmacokinetic variability described above. Pharmacokinetic variability is avoided in the method by adjusting drug dose to achieve a targeted AUC.

Olmesartan Dosing by AUC

In one aspect, the invention provides a method for olmesartan dosing by AUC. In certain embodiments, the method comprises:

(a) administering olmesartan at a first dose (e.g., under a first regiment, such as once a day) to a subject in need of hypertension therapy;

(b) determining the concentration of olmesartan the subject's blood at one or more time points (e.g., a series of time points, such as 0.5, 1, 2, 4, 8, 12, 16, hour) after administration to provide a set of olmesartan concentration/time data points;

(c) transforming the set of olmesartan concentration/time data points to provide area-under-the-curve (AUC) (i.e., the AUC resulting from the first dose); and

(d) administering olmesartan at subsequent doses (e.g., second and subsequent doses to achieve a target AUC of about 7,000 hr*ng/mL.

As noted above, for olmesartan, the target AUC is about 7,000 hr*ng/mL. In certain embodiments, the target AUC is from about 6,000 to about 8,000 hr*ng/mL. In other embodiments, the target AUC is from about 6,500 to about 7,500 hr*ng/mL. In certain embodiments, the target AUC is 7,000 hr*ng/mL +/−5%. In other embodiments, the target AUC is 7,000 hr*ng/mL +/−2%. In further embodiments, the target AUC is 7,000 hr*ng/mL +/−1%. In yet other embodiments, the target AUC is 7,000 hr*ng/mL+/−0.5%.

Because the of dose proportionality, determination of the second dose is straightforward. When the determined AUC is the same as the target AUC (about 7,000 hr*ng/mL), the second dose is the same or substantially the same as the first dose. When the determined AUC is the greater than the target, the second dose is less than the first dose by the same proportion. When the determined AUC is less than the target, the second dose is greater than the first dose by the same proportion.

In certain embodiments, the method further comprising repeating steps (a)-(d) until target AUC and/or blood pressure control is achieved.

Area-under-the-curve (AUC) is a pharmacokinetic parameter that is used in the method of the invention to dose olmesartan. As used herein, the term “area under the curve (AUC)” is the area under the curve in a plot of concentration of drug in blood plasma as a function of time. Typically, the area is calculated starting at the time the drug is administered and ending when the concentration in plasma is negligible. AUC represents the total drug exposure over time. Assuming linear pharmacodynamics with elimination rate constant K, AUC is proportional to the total amount of drug absorbed by the body (i.e., the total amount of drug that reaches the blood circulation). The proportionality constant is 1/K.

As used herein, the phrase “transforming the concentration/time data points” refers to the application of mathematical operations, formulas, theories, and/or principles (i.e., a formula for calculating AUC) to the concentrations/time data points of the individual subject to provide AUC.

The target AUC of 7,000 hr*ng/mL was determined from statistical analysis of a subject population receiving olmesartan. The target AUC is the median AUC value determined from a population of subjects receiving olmesartan at a dose of 40 mg/day (daily administration).

The term “blood pressure control” refers to maintenance of blood pressure <150 mm Hg (SBP) and <90 mm Hg (DBP) for subjects >60 years of age, and <140 mm Hg (SBP) and <90 mM Hg (DBP) for subjects <60 years of age.

In the methods of the invention, the nature of the device or method for determining the concentrations/time data points for calculating AUC is not critical. Methods and devices for determining therapeutic drug (e.g., olmesartan) concentrations are known in the art and can be used. In certain embodiments, a point-of-care device can be used to determine the concentrations and create the concentration/time data, transmit the data to a central location, and/or transmitting instructions to the patient to alter the administration.

In certain embodiments, the device and method for determining the concentrations/time data points for calculating AUC is an immunoassay assay device and method that utilizes one or more olmesartan antibodies (e.g., monoclonal antibodies) or functional fragments thereof. In certain of these embodiments, the device is a lateral flow device.

The method of the invention is therapeutically effective for delivery of olmesartan and therefore is effective for treating hypertension

The above method is also effective for treating subjects suffering from resistant hypertension. In certain embodiments, the subject treatable by the method is a subject that has been previously treated for hypertension with a three-drug regimen in which one of the three drugs (i.e., first drug) is a diuretic, and where the subject's blood pressure remained elevated above an established blood pressure goal following the three-drug regimen. In this method, the second and the third drugs of the three-drug regiment are selected from angiotensin converting enzyme inhibitors, angiotensin II receptor blockers, beta-adrenergic receptor blockers, calcium channel blockers, direct vasodilators, alpha-1-adrenergic receptor blockers, central alpha-2-adrenergic receptor agonists, and aldosterone receptor agonists. In certain embodiments, the first, second, and third drugs were administered at their highest approved dose.

The above method is also effective for treating subjects in need of combined hypertension and dyslipidemia therapy. In certain embodiments, the subject treatable by the method is a subject that is in need of treatment for hypertension and dyslipidemia. In certain embodiments of this method, olmesartan and an anti-dyslipidemia drug are individually administered. In other embodiments of this method, a single dosage form that comprises olmesartan and an anti-dyslipidemia drug (e.g., rosuvastatin or a salt thereof) is administered. In certain embodiments of this method, the single dosage form comprises olmesartan and rosuvastatin calcium.

Antihypertensive Drug Dosing by AUC

In another aspect, the invention provides a method for antihypertensive drug dosing by AUC. In certain embodiments, the method comprises:

(a) administering an antihypertensive drug at a first dose to a subject in need of hypertension therapy;

(b) determining the concentration of the antihypertensive drug the subject's blood at one or more time points after administration to provide a set of antihypertensive drug concentration/time data points;

(c) transforming the set of antihypertensive drug concentration/time data points to provide one or more pharmacokinetic parameters; and

(d) administering the antihypertensive drug at subsequent doses (e.g., second and subsequent doses) to achieve a target optimal value for the one or more pharmacokinetic parameters.

As noted above, the target pharmacokinetic parameter is the pre-determined optimal value. In certain embodiments, the target pharmacokinetic parameter is the pre-determined optimal value +/−5%. In other embodiments, the target pharmacokinetic parameter is the pre-determined optimal value +/−2%. In further embodiments, the target pharmacokinetic parameter is the pre-determined optimal value +/−1%. In yet other embodiments, the target pharmacokinetic parameter is the pre-determined optimal value+/−0.5%.

Because the of dose proportionality, determination of the second dose is straightforward. When the determined pharmacokinetic (PK) parameter is the same or substantially the same as the target PK parameter, the second dose is the same as the first dose. When the determined PK parameter is the greater than the target, the second dose is less than the first dose by the same proportion. When the determined PK parameter is less than the target, the second dose is greater than the first dose by the same proportion.

In certain embodiments, the method further comprising repeating steps (a)-(d) until the target pharmacokinetic parameter value(s) and/or blood pressure control is achieved.

Any suitable pharmacokinetic (PK) parameter or parameters can be used in accordance with this aspect of the invention, including without limiting concentration, concentration time course, peak concentration, and time after administration to peak concentration, terminal half-life, area-under-the-curve (AUC), bioavailability, absorption, distribution, metabolism, excretion, biotransformation, or a combination thereof.

As used herein, the phrase “transforming the concentration/time data points” refers to the application of mathematical operations, formulas, theories, and/or principles (e.g., a formula for calculating AUC) to the concentrations/time data points of the individual subject to provide the pharmacokinetic value (e.g., AUC).

The target pharmacokinetic value is pre-determined by statistical analysis from a population of subjects receiving the antihypertensive drug at its optimal dose. The term “optimal dose” refers to a dose (e.g., mg/day) associated with desirable drug efficacy at lower risk doses of a drug (e.g., the Cₘₐₓ range corresponding to patients experiencing high drug efficacy at a low dose) and is determined from a statistical analysis of a subject population receiving doses of the antihypertensive drug for whom there was therapeutic improvement without significant adverse drug reactions or significant side effects. Significant adverse drug reactions refer to ADRs that the subject finds intolerable, impair physiologic functions, and put the subject at risk for immobility and/or death or combinations thereof. Significant side effects refer to side effects that the subject finds intolerable, impair physiologic functions, and put the patient at risk for immobility and/or death or combinations thereof.

The term “blood pressure control” refers to maintenance of blood pressure <150 mm Hg (SBP) and <90 mm Hg (DBP) for subjects >60 years of age, and <140 mm Hg (SBP) and <90 mM Hg (DBP) for subjects <60 years of age.

As noted above, in the methods of the invention, the nature of the device or method for determining the concentrations/time data points for calculating the pharmacokinetic parameter is not critical. Methods and devices for determining therapeutic drug (e.g., antihypertensive drugs) concentrations are known in the art and can be used. In certain embodiments, a point-of-care device can be used.

In certain embodiments, the device and method for determining the concentrations/time data points for calculating the pharmacokinetic parameter is an immunoassay assay device and method that utilizes one or more olmesartan antibodies (e.g., monoclonal antibodies) or functional fragments thereof. In certain of these embodiments, the device is a lateral flow device.

The method of the invention is effective for delivering an antihypertensive drug. Representative antihypertensive drugs include angiotensin converting enzyme (ACE) inhibitors, angiotensin II receptor blockers (ARBs), beta-adrenergic receptor blockers, calcium channel blockers, direct vasodilators, alpha-1-adrenergic receptor blockers, central alpha-2-adrenergic receptor agonists, and aldosterone receptor agonists. Representative angiotensin II receptor blockers include olmesartan, losartan, candesartan, valsartan, irbesartan, telmisartan, eposartan, azilsartan and fimasartan. In certain embodiments, the antihypertensive drug is olmesartan.

The method of the invention is therapeutically effective for delivery of antihypertensive drugs and therefore is effective for treating hypertension

The above method is also effective for treating subjects suffering from resistant hypertension. In certain embodiments, the subject treatable by the method is a subject that has been previously treated for hypertension with a three-drug regimen in which one of the three drugs (i.e., first drug) is a diuretic, and where the subject's blood pressure remained elevated above an established blood pressure goal following the three-drug regimen. In this method, the second and the third drugs of the three-drug regiment are selected from angiotensin converting enzyme inhibitors, angiotensin II receptor blockers, beta-adrenergic receptor blockers, calcium channel blockers, direct vasodilators, alpha-1-adrenergic receptor blockers, central alpha-2-adrenergic receptor agonists, and aldosterone receptor agonists. In certain embodiments, the first, second, and third drugs were administered at their highest approved dose.

The above method is also effective for treating subjects in need of combined hypertension and dyslipidemia therapy. In certain embodiments, the subject treatable by the method is a subject that is in need of treatment for hypertension and dyslipidemia. In certain embodiments of this method, an antihypertensive drug and an anti-dyslipidemia drug are individually administered. In other embodiments of this method, a single dosage form that comprises an antihypertensive drug and an anti-dyslipidemia drug (e.g., rosuvastatin or a salt thereof) is administered. In certain embodiments of this method, the single dosage form comprises olmesartan and rosuvastatin.

Resistant Hypertension

Although hypertension can be controlled with lifestyle changes and drugs, uncontrolled or resistant hypertension is a significant unmet clinical need in 22 percent of the hypertensive population. Despite a wide range of drugs available for antihypertensive therapy, a segment of the patient population continues to exhibit resistance to a baseline antihypertensive therapy with one or more drugs. A particularly challenging subject population has clinically diagnosed resistant hypertension. Resistant hypertension is defined by the Seventh Report of the Joint National Committee on Prevention, Detection, Evaluation, and Treatment of High Blood Pressure (JNC 7; Chobanian et al. (2003) Hypertension 42:1206-1252) as a failure to achieve goal blood pressure in subjects who are adhering to full doses of an appropriate three-drug regimen that includes a diuretic. Further, resistant hypertension is diagnosed by many physicians on the basis of a subject's resistance to adequate, but less than full doses, of an appropriate three-drug regimen because of the risk or occurrence of adverse events associated with full doses. An “adequate” dose as prescribed by the physician can be less than or equal to a full dose of the drug. A “full” dose or “highest approved dose” is the lowest of (a) the highest dose of the drug labeled for a hypertension indication; (b) the highest usual dose of the drug prescribed according to JNC 7, BHD-IV, ESH/ESC or WHO/ISH guidelines; or (c) the highest tolerated dose of the drug in the particular subject.

As noted above, in certain embodiments, the methods of the invention are effective for treating resistant hypertension.

In certain embodiments, the subject treatable by the methods of the invention is a subject that has been previously treated for hypertension with a three-drug regimen in which one of the three drugs (i.e., first drug) is a diuretic, and where the subject's blood pressure remained elevated above an established blood pressure goal following the three-drug regimen. In this method, the second and the third drugs of the three-drug regiment are selected from angiotensin converting enzyme inhibitors, angiotensin II receptor blockers, beta-adrenergic receptor blockers, calcium channel blockers, direct vasodilators, alpha-1-adrenergic receptor blockers, central alpha-2-adrenergic receptor agonists, and aldosterone receptor agonists. In certain embodiments, the first, second, and third drugs were administered at their highest approved dose.

Hypertension and Dyslipidemia

An estimated 40 to 45 percent of hypertensive patients also suffer from dyslipidemia. Because it is considered advantageous to treat patients suffering from hypertension and dyslipidemia with a single therapeutic agent, combinations of therapeutic agents in single dose form have been developed for concomitantly treating both diseases. Combination formulations of antihypertensive and antihyperlipidemic agents are described in WO 95/26188, WO 97/37688, WO 99/11260, WO 00/45818, WO 04/062729, and WO 06/040085. One such single dose form is Caduet™, which is a clinically useful combination formulation of atorvastatin and amlodipine.

Rosuvastatin, an HMG-CoA reductase inhibitor, is useful for the treatment of hypercholesterolemia, hyperlipoproteinemia, and atherosclerosis; and rosuvastatin's calcium salt is commercially available under the designation Crestor™. Olmesartan medoxomil is useful for the treatment of essential hypertension and is commercially available under the designation Benicar™. When a single matrix formulation of rosuvastatin and olmesartan medoxomil is administered, a drug-drug interaction (DDI) between rosuvastatin and olmesartan medoxomil occurs that results in delaying the in vivo release (i.e., dissolution) of rosuvastatin calcium to the gastrointestinal fluid and thus delaying the translocation thereof to the gastrointestinal membrane, inhibiting the absorption of rosuvastatin.

Olmesartan

Olmesartan medoxomil formulations should be designed so as to exhibit high dissolution rate of olmesartan medoxomil in an in vitro comparative dissolution test, in order to obtain a bioequivalent formulation to other single formulations containing olmesartan medoxomil. In order to obtain the high in vitro dissolution rate, olmesartan medoxomil tablet comprises a preferred disintegrant, which may be one or more selected from the group consisting of low substituted hydroxypropyl cellulose, carboxymethylcellulose calcium, croscarmellose sodium, crospovidone, sodium starch glycolate, and pregelatinized starch. In one embodiment, olmesartan medoxomil tablet comprises 7.5 or more % by weight of low substituted hydroxypropyl cellulose, 5 or more % by weight of carboxymethylcellulose calcium, 15 or more % by weight of croscarmellose sodium, 10 or more % by weight of crospovidone, 5 or more % by weight of sodium starch glycolate, or 5 or more % by weight of pregelatinized starch, based on the total weight of the tablet comprising olmesartan medoxomil. In another embodiment, the compartment comprising olmesartan medoxomil comprises 7.5 to 65% by weight of low substituted hydroxypropyl cellulose, 5 to 60% by weight of carboxymethylcellulose calcium, 15 to 30% by weight of croscarmellose sodium, 10 to 40% by weight of crospovidone, 5 to 40% by weight of sodium starch glycolate, or 5 to 60% by weight of pregelatinized starch, based on the total weight of the tablet comprising olmesartan medoxomil. In a further embodiment, the tablet comprising olmesartan medoxomil comprises 7.5 to 65% by weight, preferably 10 to 60% by weight, more preferably about 20±1% by weight of low substituted hydroxypropyl cellulose, based on the total weight of the tablet comprising olmesartan medoxomil.

Olmesartan/Rosuvastatin FDC

An improved pharmaceutical composition that is a single dosage form of olmesartan medoxomil and rosuvastatin or its salts is described in WO 2013/147462. This single dosage form comprises separate compartments for each drug in which each drug is separately and independently formulated. When the single dosage form is administered the interaction to in vivo absorption is minimized and the combination formulation is bioequivalent to the single formulation of each of drugs.

In certain embodiments, the subject treatable by the methods of the invention is a subject that is in need of treatment for hypertension and dyslipidemia. In certain embodiments of this method, an antihypertensive drug and an anti-dyslipidemia drug are individually administered. In other embodiments of this method, a single dosage form that comprises an antihypertensive drug and an anti-dyslipidemia drug (e.g., rosuvastatin or a salt thereof) is administered. In certain embodiments of this method, the single dosage form comprises olmesartan and rosuvastatin.

A description of representative single dosage forms useful in the methods of the invention and methods for making the single dose forms are described in WO 2013/147462, expressly incorporated herein by reference in its entirety. Representative single dose forms useful in the method of the inventions and a method for making them are described below.

The pharmaceutical composition useful in the methods of the invention, which includes olmesartan medoxomil and rosuvastatin or its salt (e.g., rosuvastatin calcium), are formulated into a combination dosage form having separate compartments. That is, the pharmaceutical composition has a single dosage form comprising a compartment comprising olmesartan medoxomil; and a compartment comprising rosuvastatin or its salt, wherein the compartments are formulated in a separate form.

In the pharmaceutical composition, the active ingredients (i.e., olmesartan medoxomil and rosuvastatin or its salt) may be used in a therapeutically effect amount. For example, olmesartan medoxomil may be used in an amount of about 5 mg to about 80 mg, preferably about 10 mg to about 40 mg, in a unit formulation (i.e., unit dosage form).; and rosuvastatin or its salt may be used in an amount of about 2 mg to about 40 mg, preferably about 5 mg to about 20 mg, in a unit formulation (i.e., unit dosage form). The salt of rosuvastatin may be a conventional pharmaceutically acceptable salt, such as calcium salt, hydrochloride, hydrobromide, sulfate, phosphate, acetate, maleate, fumarate, lactate, tartrate, citrate, gluconate, besylate, and camsylate. Preferably, rosuvastatin calcium may be used in the present invention. The pharmaceutical composition may be administered once a day, but not limited thereto.

The pharmaceutical composition has a combination dosage form having separate compartments (i.e., a double-layered tablet form), comprising or consisting essentially of a layer comprising rosuvastatin or its salt and a layer comprising olmesartan medoxomil.

When the compartment comprising rosuvastatin or its salt includes a certain disintegrant (i.e., cellulose-type and/or povidone-type disintegrants), in a certain amount, rapid disintegration and high initial dissolution rate of rosuvastatin or its salt can be accomplished, thereby being able to obtain a combination formulation bioequivalent to the single formulation of rosuvastatin or its salt. The disintegrant may be one or more selected from the group consisting of povidone (for example, Kolidone™), crospovidone (for example, Polyplasdone™), low substituted hydroxypropyl cellulose, croscarmellose sodium, and carboxymethylcellulose calcium. Preferably, the disintegrant may be a mixture of crospovidone and croscarmellose sodium; or croscarmellose sodium. The disintegrant may be present in an amount ranging from 2 to 20% by weight, preferably from 3 to 15% by weight, based on the total weight of the compartment comprising rosuvastatin or its salt. When other disintegrants are used, the dissolution rate of rosuvastatin or its salt is decreased; and/or the amount used is increased, which may cause insufficient compression force during the compressing step, thereby leading to high friability of the resulting formulation (e.g., tablet). In addition, the use of other disintegrants brings about insufficient hardness, which may cause unwanted problems in packaging or delivery.

For olmesartan medoxomil, a combination formulation comprising rosuvastatin and olmesartan medoxomil should be designed so as to exhibit high dissolution rate of olmesartan medoxomil in an in vitro comparative dissolution test, in order to obtain a bioequivalent formulation to the single formulation containing olmesartan medoxomil. In order to obtain the high in vitro dissolution rate, the compartment comprising olmesartan medoxomil comprises a preferred disintegrant, which may be one or more selected from the group consisting of low substituted hydroxypropyl cellulose, carboxymethylcellulose calcium, croscarmellose sodium, crospovidone, sodium starch glycolate, and pregelatinized starch. In an embodiment, the compartment comprising olmesartan medoxomil comprises 7.5 or more % by weight of low substituted hydroxypropyl cellulose, 5 or more % by weight of carboxymethylcellulose calcium, 15 or more % by weight of croscarmellose sodium, 10 or more % by weight of crospovidone, 5 or more % by weight of sodium starch glycolate, or 5 or more % by weight of pregelatinized starch, based on the total weight of the compartment comprising olmesartan medoxomil. In another embodiment, the compartment comprising olmesartan medoxomil comprises 7.5 to 65% by weight of low substituted hydroxypropyl cellulose, 5 to 60% by weight of carboxymethylcellulose calcium, 15 to 30% by weight of croscarmellose sodium, 10 to 40% by weight of crospovidone, 5 to 40% by weight of sodium starch glycolate, or 5 to 60% by weight of pregelatinized starch, based on the total weight of the compartment comprising olmesartan medoxomil. In a further embodiment, the compartment comprising olmesartan medoxomil comprises 7.5 to 65% by weight, preferably 10 to 60% by weight, more preferably about 20±1% by weight of low substituted hydroxypropyl cellulose, based on the total weight of the compartment comprising olmesartan medoxomil.

The pharmaceutical composition may further comprise one or more excipients conventionally used in the field of pharmaceutics, for example a diluent (or additive), a binder, a lubricant, in addition to said disintegrant. The pharmaceutical composition may be also coated with an appropriate coating agent, such as a film-coating agent.

The diluent (or additive) includes lactose (including its hydrate), dextrin, mannitol, sorbitol, starch, microcrystalline cellulose (for example, Celphere™), silicified microcrystalline cellulose (for example, Prosolv™), calcium hydrogen phosphate (including its hydrate), anhydrous calcium hydrogen phosphate, calcium carbonate, saccharides, and a mixture thereof. The binder includes polyvinylpyrrolidone, copovidone, gelatin, starch, sucrose, methyl cellulose, ethyl cellulose, hydroxyethyl cellulose, hydroxypropyl cellulose, hydroxypropyl alkylcellulose (for example, hydroxypropyl methylcellulose), and a mixture thereof. The lubricant includes stearic acid, stearates (for example, magnesium stearate), talc, corn starch, carnauba wax, light anhydrous silicic acid, magnesium silicate, synthetic aluminum silicate, hydrogenated oil, hydrogenated oil, titanium oxide, microcrystalline cellulose, macrogol 4000 or 6000, isopropyl myristate, calcium hydrogen phosphate, and a mixture thereof. The coating agent, for example a film-coating agent, includes a conventional polymer such as Opadry™. The film-coating agent may be used in a minimum amount for providing an appropriate size of the formulation, but not limited thereto.

The pharmaceutical composition having a double-layered tablet form may be prepared by preparing granules containing rosuvastatin and granules containing olmesartan medoxomil, respectively; and then compressing the mixture thereof with a double-layer tablet-press machine. If necessary, the resulting double-layered tablet may be coated with a film-coating agent such as Opadry™. The granules containing rosuvastatin and the granules containing olmesartan medoxomil may be prepared according to dry granulation methods or wet granulation methods. For example, the granules containing rosuvastatin may be prepared according to a dry granulation method. That is, the granules containing rosuvastatin may be prepared by mixing rosuvastatin calcium, an additive (diluent), a disintegrant, and a lubricant according to a conventional method; and then granulating the mixture with e.g., a roller compactor (TF mini, Vector). And also, the granules containing olmesartan medoxomil may be prepared according to a wet granulation method. That is, the granules containing olmesartan medoxomil may be prepared by mixing olmesartan medoxomil, a binder, an additive (diluent), a disintegrant; granulating the mixture with a high speed mixer (MIC Developer-5, COMASA); and then drying and sieving the resulting granules.

Representative double-layer tablets can be prepared as described below.

Step 1. Preparation of granules containing rosuvastatin.

Rosuvastatin calcium, lactose monohydrate, Prosolv™, dibasic calcium phosphate dihydrate, crospovidone, croscarmellose sodium, light anhydrous silicic acid, and magnesium stearate (85% of the total amount used in the rosuvastatin-layer) were sieved through a 24 mesh and then mixed. The resulting mixture was granulated using a roller compactor (TF mini, Vector). The obtained granules were sieved through a 24 mesh and then mixed with magnesium stearate pre-sieved though a 35 mesh (15% of the total amount used in the rosuvastatin-layer) to prepare a rosuvastatin-containing granule mixture.

Step 2. Preparation of granules containing olmesartan medoxomil.

Olmesartan medoxomil, hydroxypropyl cellulose, lactose monohydrate, microcrystalline cellulose, and low substituted hydroxypropyl cellulose were sieved through a 24 mesh and then mixed. The resulting mixture was granulated using a high speed mixer (MIC Developer-5, COMASA). The resulting dry granules were sieved through a 24 mesh and then mixed with magnesium stearate pre-sieved though a 35 mesh and yellow iron oxide pre-sieved through a 80 mesh to prepare a olmesartan medoxomil-containing granule mixture.

Step 3. Preparation of double-layered tablets.

The rosuvastatin-containing granule mixture prepared in Step 1 and the olmesartan medoxomil-containing granule mixture prepared in Step 2 were compressed with a double-layer tablet-press machine (BB-11, RIVA) to obtain double-layered tablets. The resulting tablets were film-coated with Opadry™ in a pan coating machine (LDCS, VECTOR).

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for olmesartan dosing by AUC, comprising:
(a) administering olmesartan at a first dose to a subject in need of hypertension therapy;
(b) determining the concentration of olmesartan the subject's blood at one or more time points after administration to provide a set of olmesartan concentration/time data points;
(c) transforming the set of olmesartan concentration/time data points to provide area-under-the-curve (AUC); and
(d) administering olmesartan at subsequent doses to achieve a target AUC of about 7,000 hr*ng/mL.

2. The method of claim 1, wherein the target AUC is 7,000 hr*ng/mL +/−5%.

3. The method of claim 1, wherein the target AUC is 7,000 hr*ng/mL +/−2%.

4. The method of claim 1, wherein the target AUC is 7,000 hr*ng/mL +/−1%.

5. The method of claim 1, wherein the target AUC is 7,000 hr*ng/mL +/−0.5%.

6. The method of claim 1, wherein the second dose is substantially the same as the first dose.

7. The method of claim 1, wherein the second dose is greater than the first dose.

8. The method of claim 1, wherein the second dose is less than the first dose.

9. The method of claim 1 further comprising repeating steps (a)-(d) until blood pressure control is achieved.

10. The method of claim 1, wherein the subject is in need of treatment for hypertension and dyslipidemia, and the method comprises administration of a single dosage form that comprises olmesartan an anti-dyslipidemia drug.

11. The method of claim 10, wherein single dosage form comprises olmesartan and rosuvastatin.

12. The method of claim 1, wherein the hypertension is resistant hypertension.

13. The method of claim 12, wherein the subject was previously treated for hypertension with a three-drug regimen comprising a first drug, a second drug, and a third drug, wherein the first drug is a diuretic, and wherein the subject's blood pressure remained elevated above an established blood pressure goal following the three-drug regimen.

14. The method of claim 13, wherein the second and the third drugs are selected from the group consisting of angiotensin converting enzyme inhibitors, angiotensin II receptor blockers, beta-adrenergic receptor blockers, calcium channel blockers, direct vasodilators, alpha-1-adrenergic receptor blockers, central alpha-2-adrenergic receptor agonists, and aldosterone receptor agonists.

15. The method of claim 13, wherein the first, second, and third drugs were administered at a highest approved dose.

16. A method for antihypertensive drug dosing by one or more pharmacokinetic parameters, comprising:
(a) administering an antihypertensive drug at a first dose to a subject in need of hypertension therapy;
(b) determining the concentration of the antihypertensive drug the subject's blood at one or more time points after administration to provide a set of antihypertensive drug concentration/time data points;
(c) transforming the set of antihypertensive drug concentration/time data points to provide one or more pharmacokinetic parameters; and
(d) administering the antihypertensive drug at subsequent doses to achieve a target optimal value for the one or more pharmacokinetic parameters.

17. The method of claim 16, wherein the one or more pharmacokinetic parameters is selected from the group consisting of concentration time course, peak concentration (Cₘₐₓ), and time after administration to peak concentration, terminal half-life, area-under-the-curve (AUC), bioavailability, absorption, distribution, metabolism, excretion, biotransformation, and combinations thereof.

18. The method of claim 16, wherein the one or more pharmacokinetic parameters is area-under-the-curve (AUC).

19. The method of claim 16, wherein the target optimal value is +/−5% of the target optimal value.

20. The method of claim 16 further comprising repeating steps (a)-(d) until blood pressure control is achieved.

21. The method of claim 16, wherein the antihypertensive drug is selected from the group consisting of an angiotensin converting enzyme (ACE) inhibitor, an angiotensin II receptor blocker (ARB), a beta-adrenergic receptor blocker, a calcium channel blocker, a direct vasodilator (e.g., thiazide diuretic), an alpha-1-adrenergic receptor blocker, a central alpha-2-adrenergic receptor agonist, and an aldosterone receptor agonist.

22. The method of claim 16, wherein the antihypertensive drug is an angiotensin II receptor blocker (ARB) selected from the group consisting of olmesartan, losartan, candesartan, valsartan, irbesartan, telmisartan, eposartan, azilsartan and fimasartan.

23. The method of claim 16, wherein the antihypertensive drug is olmesartan.

24. The method of claim 16, wherein the subject is in need of treatment for hypertension and dyslipidemia, and the method comprises administration of an antihypertensive drug and an anti-dyslipidemia drug.

25. The method of 16, wherein the subject is in need of treatment for hypertension and dyslipidemia, and the method comprises administration of a single dosage form that comprises an antihypertensive drug and an anti-dyslipidemia drug.

26. The method of claim 25, wherein single dosage form comprises olmesartan and rosuvastatin.

27. The method of claim 16, wherein the hypertension is resistant hypertension.

28. The method claim 27, wherein the subject was previously treated for hypertension with a three-drug regimen comprising a first drug, a second drug, and a third drug, wherein the first drug is a diuretic, and wherein the subject's blood pressure remained elevated above an established blood pressure goal following the three-drug regimen.

29. The method of claim 28, wherein the second and the third drugs are selected from the group consisting of angiotensin converting enzyme inhibitors, angiotensin II receptor blockers, beta-adrenergic receptor blockers, calcium channel blockers, direct vasodilators, alpha-1-adrenergic receptor blockers, central alpha-2-adrenergic receptor agonists, and aldosterone receptor agonists.

30. The method of claim 28, wherein the first, second, and third drugs were administered at a highest approved dose.
